(19) United States
(12) Patent Application Publication
(10) Pub. No.: US 2017/0119356 A1
(43) Pub. Date: May 4, 2017

(54) METHODS AND SYSTEMS FOR A VELOCITY THRESHOLD ULTRASOUND IMAGE

(71) Applicant: General Electric Company, Schenectady, NY (US)

(72) Inventors: Josef Steininger, Zipf (AT); Helmut Brandl, Zipf (AT)

(21) Appl. No.: 14/928,176

(22) Filed: Oct. 30, 2015

(51) Int. Cl. A61B 8/08; A61B 8/06; A61B 8/00

(52) U.S. Cl. CPC A61B 8/5207; A61B 8/469; A61B 8/488; A61B 8/483; A61B 8/06; A61B 8/463; A61B 8/5223; A61B 8/5269

(57) ABSTRACT

Systems and methods are provided for generating an ultrasound image. The systems and methods collect ultrasound data for a volumetric region of interest (ROI). The ultrasound data includes color flow data and B-mode image data. The systems and methods further include a Doppler signal power and a flow velocity based on the color flow data corresponding to pixels within a first set of pixel data, and generate a second set of pixel data based on the B-mode image data. The systems and methods further identify a select pixel data set from the first set of pixels data and the second set of pixel data based on the Doppler signal power and the flow velocity. The select pixel data forms an ultrasound image of the ROI. The systems and methods further display the ultrasound image on a display.

### FIELD

Embodiments described herein generally relate to providing an ultrasound image of a diagnostic medical imaging system based on a velocity threshold applied to color flow data.

### BACKGROUND OF THE INVENTION

Diagnostic medical imaging systems typically include a scan portion and a control portion having a display. For example, ultrasound imaging systems usually include ultrasound scanning devices, such as ultrasound probes having transducers that are connected to an ultrasound system to control the acquisition of ultrasound data by performing various ultrasound scans (e.g., imaging a volume or body). The ultrasound systems are controllable to operate in different modes of operation to perform the different scans. The signals received at the probe are then communicated and processed at a back end. When the scan is complete, the ultrasound data may be stored on a patient archive communication system (PACS) for retrospective examination.

Conventional ultrasound imaging systems include a set of imaging modes, such as B-mode, color flow, and spectral Doppler imaging. In the B-mode, such ultrasound imaging systems create two dimensional images of tissue structure in which the brightness of a pixel is based on the intensity of the echo return. For color flow imaging, the general movement or velocity of fluid (e.g., blood) or tissue is imaged in a flow image determined from a Doppler shift between transmitted and return ultrasound pulses. The flow image is conventionally displayed as an overlay or mapped on a B-mode image to view both an anatomical image and a flow velocity. Conventionally, the overlaid image is formed by replacing B-mode pixels with flow image pixels that are only above a set signal power.

In daily routine scans, the user will view various flow patterns within the overlaid image, by adjusting ranges of velocities viewed within the overlaid image. However, to filter the velocities for the corresponding ranges of velocities conventional systems will adjust the intensity of the flow image pixels to black. Thus, the anatomical information provided by the B-mode pixels are continually lost by the replaced flow image pixels through the changes in velocities.

### BRIEF DESCRIPTION OF THE INVENTION

In one embodiment, a method is provided for generating an ultrasound image. The method may include collecting ultrasound data for a volumetric region of interest (ROI). The ultrasound data includes color flow data and B-mode image data. The method may further include calculating a Doppler signal power and a flow velocity based on the color flow data corresponding to pixels within a first set of pixel data, and generating a second set of pixel data based on the B-mode image data. The method may also include identifying a select pixel data set from the first set of pixel data and the second set of pixel data based on the Doppler signal power and the flow velocity. The select pixel data forming an ultrasound image of the ROI. The method may further include displaying the ultrasound image on a display.

In another embodiment, an ultrasound imaging system is provided. The ultrasound imaging system may include an ultrasound probe configured to acquire ultrasound data of a patient, a display, and a memory configured to store programmed instructions. The ultrasound data may include color flow data and B-mode image data. The ultrasound imaging system may also include one or more processors configured to execute the programmed instructions stored in the memory. The one or more processors when executing the programmed instructions perform one or more operations. The one or more processors may collect the ultrasound data from the ultrasound probe for a volumetric region of interest (ROI), calculate a Doppler signal power and a flow velocity based on the color flow data corresponding to pixels within a first set of pixel data, generate a second set of pixel data based on the B-mode image data, identify a select pixel data set from the first set of pixel data and the second set of pixel data based on the Doppler signal power and the flow velocity, and display an ultrasound image on the display. The select pixel data forming the ultrasound image of the ROI.

In another embodiment, a tangible and non-transitory computer readable medium may include one or more computer software modules configured to direct one or more processors. The one or more computer software modules may be configured to direct the one or more processors to collect ultrasound data for a volumetric region of interest (ROI). The ultrasound data includes color flow data and B-mode image data. The one or more computer software modules may further be configured to direct the one or more processors to calculate a Doppler signal power and a flow velocity based on the color flow data corresponding to pixels within a first set of pixel data, generate a second set of pixel data based on the B-mode image data, and identify a select pixel data set from the first set of pixel data and the second set of pixel data based on the Doppler signal power and the flow velocity. The select pixel data forms an ultrasound image of the ROI. The one or more computer software modules may further be configured to direct the one or more processors to display the ultrasound image on the display.

### BRIEF DESCRIPTION OF THE DRAWINGS

FIG. 1 illustrates a schematic block diagram of an ultrasound imaging system, in accordance with an embodiment.

FIG. 2 is an illustration of a simplified block diagram of a controller circuit of the ultrasound imaging system of FIG. 1, in accordance with an embodiment.

FIG. 3 illustrate a flowchart of a method for providing an ultrasound image, in accordance with an embodiment.

FIG. 4 illustrates a flowchart of a method for identifying a select pixel data set, in accordance with an embodiment.

FIG. 5 illustrates a schematic of a portion of a first set of pixel data, a second set of pixel data, in accordance with an embodiment.

FIG. 6 is a graphical illustration of Doppler signal power of a subset of the pixels of the first set of pixel data shown in FIG. 5, in accordance with an embodiment.

FIG. 7 is a graphical illustration representing of flow velocity of a subset of the pixels of the first set of pixel data shown in FIG. 5, in accordance with an embodiment.

FIG. 8 illustrates a schematic of a portion of the first set of pixel data, the second set of pixel data of FIG. 5 and a select pixel data, in accordance with an embodiment.

FIG. 9 illustrates an ultrasound image, in accordance with an embodiment.

FIG. 10 illustrates a 3D capable miniaturized ultrasound system having a probe that may be configured to acquire 3D ultrasonic data or multi-plane ultrasonic data.

FIG. 11 illustrates a hand carried or pocket-sized ultrasound imaging system wherein the display and user interface form a single unit.

FIG. 12 illustrates an ultrasound imaging system provided on a movable base.

### DETAILED DESCRIPTION OF THE INVENTION

The following detailed description of certain embodiments will be better understood when read in conjunction with the appended drawings. To the extent that the figures illustrate diagrams of the functional modules of various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or a block of random access memory, hard disk, or the like). Similarly, the programs may be stand-alone programs, may be incorporated as subroutines in an operating system, may be functions in an installed software package, and the like. It should be understood that the various embodiments are not limited to the arrangements and instrumentality shown in the drawings.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising” or “having” an element or a plurality of elements having a particular property may include additional elements not having that property.

Various embodiments provide systems and methods for providing a current color flow mapping presentation of an ultrasound image. In operation, the provided systems and methods select which pixels or voxels to select from a color flow image and a B-mode image to form an ultrasound image. The selection of the pixels or voxels is based on a calculated color flow power or Doppler power and a flow velocity corresponding to and/or represented by the pixels or voxels of the color flow image.

A technical effect of at least one embodiment described herein allows a user to appreciate flow patterns by adjusting velocity settings without losing structural information of the region of interest.

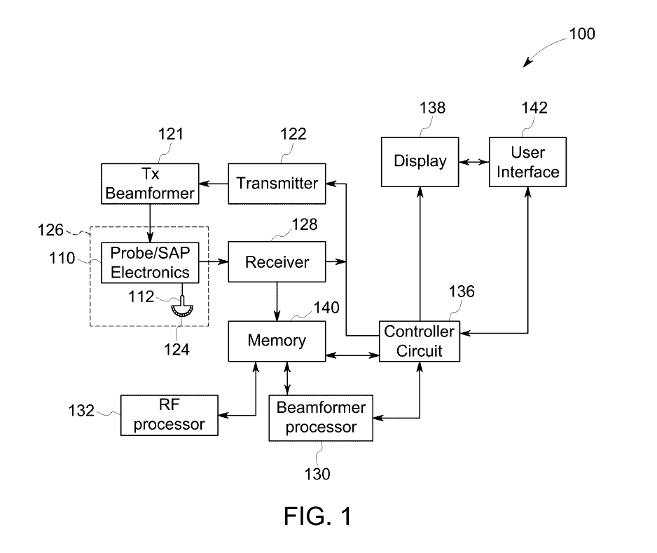

FIG. 1 is a schematic diagram of a diagnostic medical imaging system, specifically, an ultrasound imaging system 100. The ultrasound imaging system 100 includes an ultrasound probe 126 having a transmitter 122 and probe/SAP electronics 110. The ultrasound probe 126 may be configured to acquire ultrasound data or information from a region of interest (e.g., organ, blood vessel, heart) of the patient. The ultrasound probe 126 is communicatively coupled to the controller circuit 136 via the transmitter 122. The transmitter 122 transmits a signal to a transmit beamformer 121 based on acquisition settings received by the user. The signal transmitted by the transmitter 122 in turn drives the transducer elements 124 within the transducer array 112. The transducer elements 124 emit pulsed ultrasonic signals into a patient (e.g., a body). A variety of a geometries and configurations may be used for the array 112. Further, the array 112 of transducer elements 124 may be provided as part of, for example, different types of ultrasound probes.

The acquisition settings may define an amplitude, pulse width, frequency, and/or the like of the ultrasonic pulses emitted by the transducer elements 124. The acquisition settings may be adjusted by the user by selecting a gain setting, power, time gain compensation (TGC), resolution, and/or the like from the user interface 142.

The transducer elements 124, for example piezoelectric crystals, emit pulsed ultrasonic signals into a body (e.g., patient) or volume corresponding to the acquisition settings along one or more scan planes. The ultrasonic signals may include, for example, one or more reference pulses, one or more pushing pulses (e.g., shear-waves), and/or one or more pulsed wave Doppler pulses. At least a portion of the pulsed ultrasonic signals back-scatter from a region of interest (ROI) (e.g., heart, left ventricular outflow tract, breast tissues, liver tissues, cardiac tissues, prostate tissues, and the like) to produce echoes. The echoes are delayed in time and/or frequency according to a depth or movement, and are received by the transducer elements 124 within the transducer array 112. The ultrasonic signals may be used for imaging, for generating and/or tracking shear-waves, for measuring changes in position or velocity within the ROI (e.g., flow velocity, movement of blood cells), differences in compression displacement of the tissue (e.g., strain), and/or for therapy, among other uses. For example, the probe 126 may deliver low energy pulses during imaging and tracking, medium to high energy pulses to generate shear-waves, and high energy pulses during therapy.

The transducer array 112 may have a variety of array geometries and configurations for the transducer elements 124 which may be provided as part of, for example, different types of ultrasound probes 126. The probe/SAP electronics 110 may be used to control the switching of the transducer elements 124. The probe/SAP electronics 110 may also be used to group the transducer elements 124 into one or more sub-apertures.

The transducer elements 124 convert the received echo signals into electrical signals which may be received by a receiver 128. The receiver 128 may include one or more amplifiers, an analog to digital converter (ADC), and/or the like. The receiver 128 may be configured to amplify the received echo signals after proper gain compensation and convert these received analog signals from each transducer element 124 to digitized signals sampled uniformly in time. The digitized signals representing the received echoes are stored on memory 140, temporarily. The digitized signals correspond to the backscattered waves receives by each transducer element 124 at various times. After digitization, the signals still may preserve the amplitude, frequency, phase information of the backscatter waves.

Optionally, the controller circuit 136 may retrieve the digitized signals stored in the memory 140 to prepare for the beamformer processor 130. For example, the controller circuit 136 may convert the digitized signals to baseband signals or compressing the digitized signals.

The beamformer processor 130 may include one or more processors. Optionally, the beamformer processor 130 may include a central controller circuit (CPU), one or more microprocessors, or any other electronic component capable of processing inputted data according to specific logical instructions. Additionally or alternatively, the beamformer processor 130 may execute instructions stored on a tangible and non-transitory computer readable medium (e.g., the memory 140) for beamforming calculations using any suitable beamforming method such as adaptive beamforming, synthetic transmit focus, aberration correction, synthetic aperture, clutter reduction and/or adaptive noise control, and/or the like.

The beamformer processor 130 may further perform filtering and decimation, such that only the digitized signals corresponding to relevant signal bandwidth is used, prior to beamforming of the digitized data. For example, the beamformer processor 130 may form packets of the digitized data based on scanning parameters corresponding to focal zones, expanding aperture, imaging mode (B-mode, color flow), and/or the like. The scanning parameters may define channels and time slots of the digitized data that may be beamformed, with the remaining channels or time slots of digitized data that may not be communicated for processing (e.g., discarded).

The beamformer processor 130 performs beamforming on the digitized signals and outputs a radio frequency (RF) signal. The RF signal is then provided to an RF processor 132 that processes the RF signal. The RF processor 132 may generate different ultrasound image data types, e.g. B-mode, color Doppler (velocity/power/variance), tissue Doppler (velocity), and Doppler energy, for multiple scan planes or different scanning patterns. For example, the RF processor 132 may generate tissue Doppler data for multi-scan planes. The RF processor 132 gathers the information (e.g. I/Q, B-mode, color Doppler, tissue Doppler, and Doppler energy information) related to multiple data slices and stores the data information, which may include time stamp and orientation/rotation information, in the memory 140.

Alternatively, the RF processor 132 may include a complex demodulator (not shown) that demodulates the RF signal to form IQ data pairs representative of the echo signals. The RF or IQ signal data may then be provided directly to the memory 140 for storage (e.g., temporary storage). Optionally, the output of the beamformer processor 130 may be passed directly to the controller circuit 136.

The controller circuit 136 may be configured to process the acquired ultrasound data (e.g., RF signal data or IQ data pairs) and prepare frames of ultrasound image data for display on the display 138. The controller circuit 136 may include one or more processors. Optionally, the controller circuit 136 may include a central controller circuit (CPU), one or more microprocessors, a graphics controller circuit (GPU), or any other electronic component capable of processing inputted data according to specific logical instructions. Having the controller circuit 136 that includes a GPU may be advantageous for computation-intensive operations, such as volume-rendering. Additionally or alternatively, the controller circuit 136 may execute instructions stored on a tangible and non-transitory computer readable medium (e.g., the memory 140).

The controller circuit 136 is configured to perform one or more processing operations according to a plurality of selectable ultrasound modalities on the acquired ultrasound data, adjust or define the ultrasonic pulses emitted from the transducer elements 124, adjust one or more image display settings of components (e.g., ultrasound images, interface components, positioning regions of interest) displayed on the display 138, and other operations as described herein. Acquired ultrasound data may be processed in real-time by the controller circuit 136 during a scanning or therapy session as the echo signals are received. Additionally or alternatively, the ultrasound data may be stored temporarily in the memory 140 during a scanning session and processed in less than real-time in a live or off-line operation.

The memory 140 may be used for storing processed frames of acquired ultrasound data that are not scheduled to be displayed immediately or to store post-processed images (e.g., shear-wave images, strain images), firmware or software corresponding to, for example, a graphical user interface, one or more default image display settings, programmed instructions (e.g., for the controller circuit 136, the beamformer processor 130, the RF processor 132), and/or the like. The memory 140 may be a tangible and non-transitory computer readable medium such as flash memory, RAM, ROM, EEPROM, and/or the like.

The memory 140 may store 3D ultrasound image data sets of the ultrasound data, where such 3D ultrasound image data sets are accessed to present 2D and 3D images. For example, a 3D ultrasound image data set may be mapped into the corresponding memory 140, as well as one or more reference planes. The processing of the ultrasound data, including the ultrasound image data sets, may be based in part on user inputs, for example, user selections received at the user interface 142.

The controller circuit 136 is operably coupled to a display 138 and a user interface 142. The display 138 may include one or more liquid crystal displays (e.g., light emitting diode (LED) backlight), organic light emitting diode (OLED) displays, plasma displays, CRT displays, and/or the like. The display 138 may display patient information, ultrasound images and/or videos, components of a display interface, one or more 2D, 3D, or 4D ultrasound image data sets from ultrasound data stored in the memory 140 or currently being acquired, measurements, diagnosis, treatment information, and/or the like received by the display 138 from the controller circuit 136.

The user interface 142 controls operations of the controller circuit 136 and is configured to receive inputs from the user. The user interface 142 may include a keyboard, a mouse, a touchpad, one or more physical buttons, and/or the like. Optionally, the display 138 may be a touch screen display, which includes at least a portion of the user interface 142.

For example, a portion of the user interface 142 may correspond to a graphical user interface (GUI) generated by the controller circuit 136 shown on the display. The GUI may include one or more interface components that may be selected, manipulated, and/or activated by the user operating the user interface 142 (e.g., touch screen, keyboard, mouse). The interface components may be presented in varying shapes and colors, such as a graphical or selectable icon, a slide bar, a cursor, and/or the like. Optionally, one or more interface components may include text or symbols, such as a drop-down menu, a toolbar, a menu bar, a title bar, a window (e.g., a pop-up window) and/or the like. Additionally or alternatively, one or more interface components may indicate areas within the GUI for entering or editing information (e.g., patient information, user information, diagnostic information), such as a text box, a text field, and/or the like.

In various embodiments, the interface components may perform various functions when selected, such as measurement functions, editing functions, database access/search functions, diagnostic functions, controlling acquisition settings, and/or system settings for the ultrasound imaging system 100 performed by the controller circuit 136.

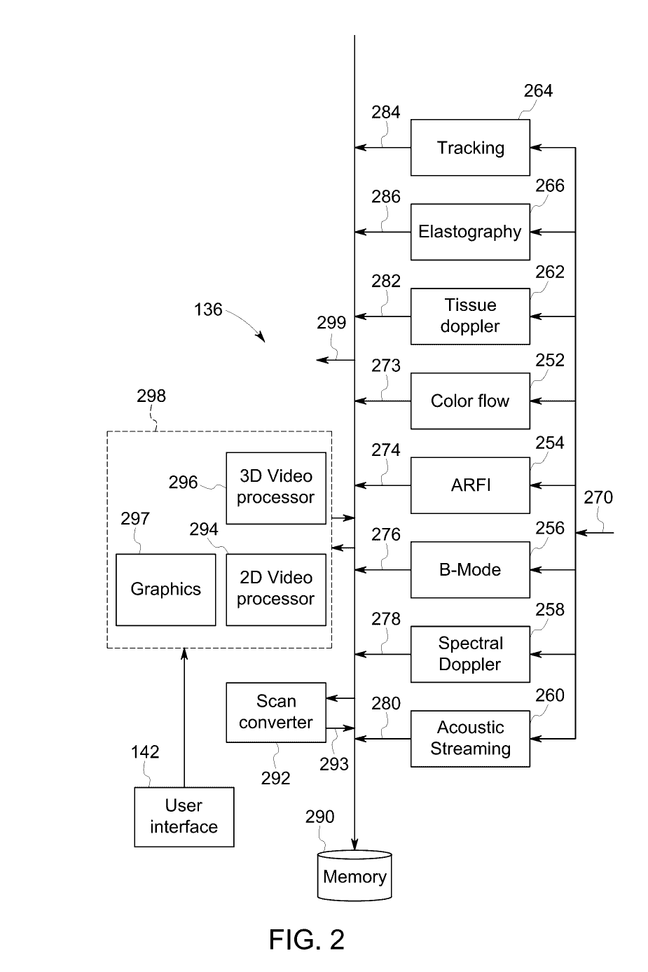

FIG. 2 is an exemplary block diagram of the controller circuit 136. The controller circuit 136 is illustrated in FIG. 2 conceptually as a collection of circuits and/or software modules, but may be implemented utilizing any combination of dedicated hardware boards, DSPs, one or more processors, FPGAs, ASICs, a tangible and non-transitory computer readable medium configured to direct one or more processors, and/or the like.

The circuits 252-266 perform mid-processor operations representing one or more software features of the ultrasound imaging system 100. The controller circuit 136 may receive ultrasound data 270 in one of several forms. In the embodiment of FIG. 1, the received ultrasound data 270 constitutes IQ data pairs representing the real and imaginary components associated with each data sample of the digitized signals. The IQ data pairs are provided to one or more circuits, for example, a color-flow circuit 252, an acoustic radiation force imaging (ARFI) circuit 254, a B-mode circuit 256, a spectral Doppler circuit 258, an acoustic streaming circuit 260, a tissue Doppler circuit 262, a tracking circuit 264, and an electrography circuit 266. Other circuits may be included, such as an M-mode circuit, power Doppler circuit, among others. However, embodiments described herein are not limited to processing IQ data pairs. For example, processing may be done with RF data and/or using other methods. Furthermore, data may be processed through multiple circuits.

Each of circuits 252-266 is configured to process the IQ data pairs in a corresponding manner to generate, respectively, color flow data 273, ARFI data 274, B-mode data 276, spectral Doppler data 278, acoustic streaming data 280, tissue Doppler data 282, tracking data 284, electrography data 286 (e.g., strain data, shear-wave data), among others, all of which may be stored in a memory 290 (or the memory 140 shown in FIG. 1) temporarily before subsequent processing. The data 273-286 may be stored, for example, as sets of vector data values, where each set defines an individual ultrasound image frame. The vector data values are generally organized based on the polar coordinate system.

A scan converter circuit 292 accesses and obtains from the memory 290 the vector data values associated with an image frame and converts the set of vector data values to Cartesian coordinates to generate an ultrasound image frame 293 formatted for display. The ultrasound image frames 293 generated by the scan converter circuit 292 may be provided back to the memory 290 for subsequent processing or may be provided to the memory 140. Once the scan converter circuit 292 generates the ultrasound image frames 293 associated with the data, the image frames may be stored in the memory 290 or communicated over a bus 299 to a database (not shown), the memory 140, and/or to other processors (not shown).

The display circuit 298 accesses and obtains one or more of the image frames from the memory 290 and/or the memory 140 over the bus 299 to display the images onto the display 138. The display circuit 298 receives user input from the user interface 142 selecting one or image frames to be displayed that are stored on memory (e.g., the memory 290) and/or selecting a display layout or configuration for the image frames.

The display circuit 298 may include a 2D video processor circuit 294. The 2D video processor circuit 294 may be used to combine one or more of the frames generated from the different types of ultrasound information. Successive frames of images may be stored as a cine loop (4D images) in the memory 290 or memory 140. The cine loop represents a first in, first out circular image buffer to capture image data that is displayed in real-time to the user. The user may freeze the cine loop by entering a freeze command at the user interface 142.

The display circuit 298 may include a 3D processor circuit 296. The 3D processor circuit 296 may access the memory 290 to obtain spatially consecutive groups of ultrasound image frames and to generate three-dimensional image representations thereof, such as through volume rendering or surface rendering algorithms as are known. The three-dimensional images may be generated utilizing various imaging techniques, such as ray-casting, maximum intensity pixel or voxel projection and the like.

The display circuit 298 may include a graphic circuit 297. The graphic circuit 297 may access the memory 290 to obtain groups of ultrasound image frames and the ROI data acquisition locations that have been stored or that are currently being acquired. The graphic circuit 297 may generate images that include the images of the ROI and a graphical representation positioned (e.g., overlaid) onto the images of the ROI. The graphical representation may represent an outline of a treatment space, the focal point or region of the therapy beam, a path taken by the focal region within the treatment space, a probe used during the session, the ROI data acquisition location, and the like. Graphical representations may also be used to indicate the progress of the therapy session. The graphical representations may be generated using a saved graphical image or drawing (e.g., computer graphic generated drawing), or the graphical representation may be directly drawn by the user onto the image using a GUI of the user interface 142.

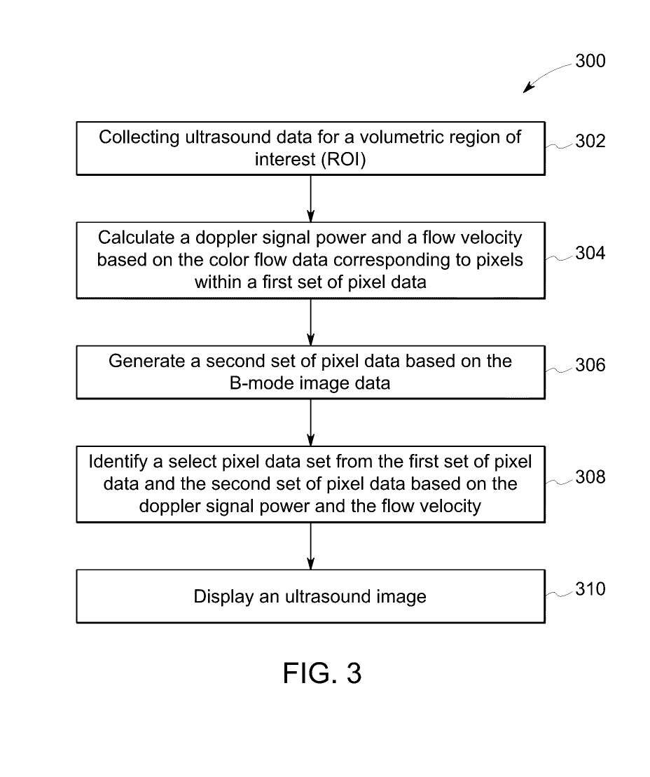

In connection with FIG. 3, the user may select an interface component corresponding to generate color mapped ultrasound image, which includes a color flow image based on color data 273 overlaid on a B-mode image based on B-mode data 276 using the user interface 142. When the interface component is selected, the controller circuit 136 may perform one or more of the operations described in connection with method 300.

FIG. 3 illustrate a flowchart of a method 300 for providing an ultrasound image, in accordance with various embodiments described herein. The method 300, for example, may employ structures or aspects of various embodiments (e.g., systems and/or methods) discussed herein. In various embodiments, certain steps (or operations) may be omitted or added, certain steps may be combined, certain steps may be performed simultaneously, certain steps may be performed concurrently, certain steps may be split into multiple steps, certain steps may be performed in a different order, or certain steps or series of steps may be re-performed in an iterative fashion. In various embodiments, portions, aspects, and/or variations of the method 300 may be used as one or more algorithms to direct hardware to perform one or more operations described herein. It should be noted, other methods may be used, in accordance with embodiments herein.

One or more methods may (i) collect ultrasound data for a volumetric region of interest (ROI), (ii) calculate a Doppler signal power and flow velocity based on the color flow data corresponding to pixels within a first set of pixel data, (iii) generate a second set of pixel data based on the B-mode image data, (iv) identify a select pixel data set from the first set of pixel data and the second set of pixel data based on the Doppler signal power and the flow velocity, and (v) display the ultrasound image on a display.

Beginning at 302, the controller circuit 136 may collect ultrasound data for a volumetric region of interest (ROI). The ROI may correspond to a cardiac structure, such as a heart, left ventricle, right ventricle, left ventricular outflow tract, and/or the like. The user may position the ultrasound probe 126 (FIG. 1) to align the transducer array 112, for example, at an abdominal view, a four-chamber, five-chamber, short-axis and three-vessel view of the heart.

Ultrasound acquisition settings may configure the ultrasound probe 126 to collect ultrasound data of the ROI. The ultrasound acquisition settings may be defined based on signals received by the user interface 142. For example, the user may select one or more interface components displayed on the GUI and/or select keystrokes corresponding to one or more of the circuits 260-266, such as an interface component corresponding to color flow mapping, using the user interface 142 (FIG. 1).

The ultrasound data includes color flow data and B-mode image data. For example, the controller circuit 136 may adjust the ultrasound acquisition settings (e.g., the gain, power, time gain compensation (TGC), resolution, and/or the like of the ultrasound probe 126) and process received ultrasound imaging data. Based on the ultrasound acquisition settings, the transducer elements 124 may emit ultrasonic pulses over a period of time, of which, at least a portion may be for color flow imaging and another portion for B-mode imaging. In various embodiments, the ultrasonic pulses corresponding to the color flow imaging and the B-mode imaging may be interleaved.

For example, the transducer elements 124 may transmit pulse sequences corresponding to color flow imaging or B-mode imaging. The pulse sequences may include tone bursts of a length P, and are fired repeatedly at a pulse repetition frequency (PRF) focused at a focal position. In operation, the pulse sequences corresponding to the color flow imaging may be interposed between the pulse sequences corresponding to the B-mode imaging. For example, a color flow imaging pulse sequence may be transmitted subsequent to and preceding a B-mode imaging pulse sequence.

At least a portion of the ultrasonic pulses are backscattered by the tissue of the ROI and received by the receiver 128, which converts the received echo signals into digitized signals. The digitized signals, as described herein, are beamformed by the beamformer processor 130 and formed into IQ data pairs (e.g., the ultrasound data 270) representative of the echo signals by the RF processor 132, and are received as ultrasound data 270 by the controller circuit 136, which may be formed into B-mode data 276 and color flow data 273 by the B-mode circuit 256 and the color flow circuit 252, respectively.

At 304, the controller circuit 136 calculates a Doppler signal power and a flow velocity based on the color flow data corresponding to pixels within a first set of pixel data. The first set of pixel data may be an array of pixels forming one or more frames corresponding to color flow images generated by the controller circuit 136. Each of the pixels of the first set of pixel data may represent and/or include information corresponding to flow velocity and Doppler signal power.

In operation, the color flow circuit 252 or generally the controller circuit 136 may receive the ultrasound data 270 stored in the memory 140 collected in response to the pulse sequences of the color flow imaging. The color flow circuit 252 may perform filtering (e.g., wall filter, high pass filter, band-pass filter) to remove or reject stationary or slow-moving tissue from the ultrasound data 270 to decrease a processing load on the controller circuit 136. The color flow circuit 252 may convert the received ultrasound data 270 into intermediate parameters N, D, and R(0) for locations or range cells of the ROI, which may correspond to resulting pixels of the first pixel data set. R(0) may be an estimate of the returned power or Doppler signal power of the received echo signals calculated by the filtered ultrasound data 270, for example, the ultrasound data 270 may be IQ data pairs calculated by the RF processors 132 stored in the memory 140, as shown in Equation 1. R(0) is approximated as a finite sum over a number of pulse sequences (e.g., represented by the variable M) at a position within the ROI.

$$R ( 0 ) = {\sum }_{i = 1}^{M - 1} \frac{( {I}_{i}^{2} + {Q}_{i}^{2} + {I}_{i + 1}^{2} + {Q}_{i + 1}^{2} )}{2} \qquad ( Equation 1 )$$

The color flow circuit 252 calculates a flow velocity (e.g., movement of the tissue with respect to the ultrasound probe 126, blood flow) along the one more scan planes of the ultrasound probe 126 for multiple vector positions and multiple range gates within the tissue based on phase shifts of the digitized signals with respect to the transmitted ultrasound pulses. The flow velocity may be based on the variables N and D calculated by the color flow circuit 252 as shown below in Equations 2 and 3 to determine a phase shift as shown in Equation 4. The variable T corresponding to the pulse repetition time between each pulse in the pulse sequence corresponding to the color flow imaging.

$$\begin{matrix}N = {\sum }_{i = 1}^{M - 1} ( {I}_{i} {Q}_{i + 1} - {I}_{i + 1} {Q}_{i} ) & ( Equation 2 ) \\ D = {\sum }_{i = 1}^{M - 1} ( {I}_{i} {I}_{i + 1} + {Q}_{i} {Q}_{i + 1} ) & ( Equation 3 ) \\ φ ( R ( T ) ) = {tan}^{- 1} [ \frac{N}{D} ] & ( Equation 4 )\end{matrix}$$

The color flow circuit 252 may use the phase to calculate a mean Doppler frequency, as shown in Equation 5, which is proportional to the flow velocity as shown in the Doppler shift equation of Equation 6. The angle represented by θ is the Doppler angle.

$$\begin{matrix}\overline{f} = \frac{1}{2 π T} ( φ ( R ( T ) ) ) & ( Equation 5 ) \\ \overline{v} = \frac{\overline{f}}{{f}_{0}} \cdot \frac{c}{2 cos θ} & ( Equation 6 )\end{matrix}$$

The color flow circuit 252 may calculate a Doppler signal power and flow velocity for each of the pixels within the first set of pixel data. For example, the flow velocity information may be included within vector data values of the color flow data 273 defining individual frames of the color flow image corresponding to the first set of pixel data, which is converted to pixels by the scan converter 292. The vector data values may include pixel color information, such as red and blue, to represent a speed and a direction (e.g., with respect to the ultrasound probe 126) of the flow velocity.

At 306, the controller circuit 136 generates a second set of pixel data based on the B-mode image data. In various embodiments, the B-mode ultrasound image may be a subharmonic or fundamental image based on the received echo signals resulting by the pulse sequences corresponding to the B-mode imaging transmitted by the transducer elements 124. The received signals associated with the pulse sequences may be included in the ultrasound data received by the B-mode circuit 256 as the ultrasound data 270. Optionally, the B-mode circuit 256 may include a filter, such as a bandpass filter centered at a second harmonic or subharmonic frequency relative to the pulses of the pulse sequences. The B-mode circuit 256 or generally the controller circuit 136 may calculate vector data values from the received signals for the B-mode ultrasound image corresponding to the second set of pixel data, which are stored in the memory 290.

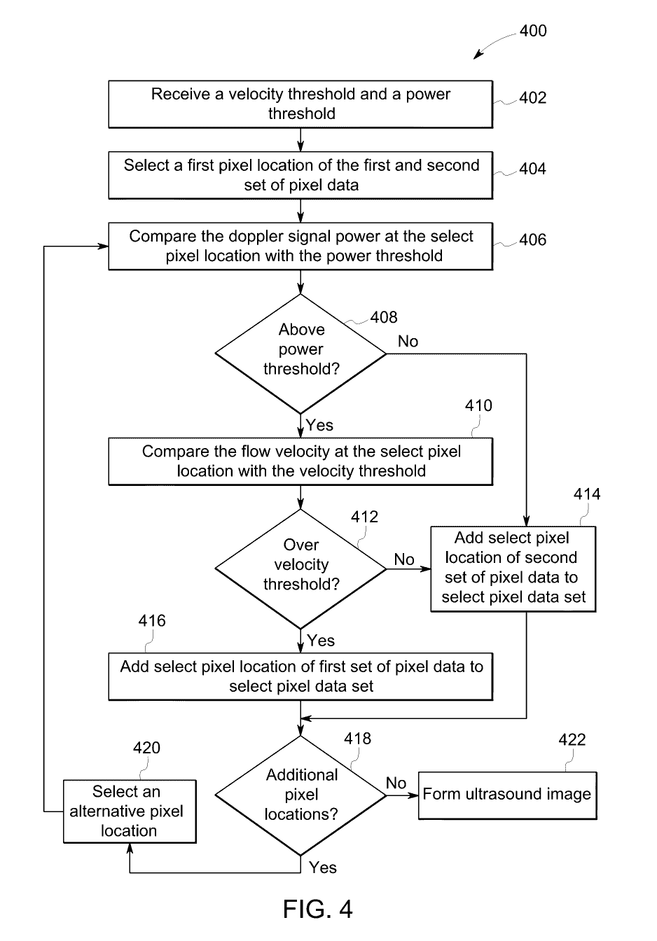

At 308, the controller circuit 136 identifies a select pixel data set from the first set of pixel data and the second set of pixel data based on the Doppler signal power and the flow velocity. The select pixel data set may include pixels of the first set of pixel data and the second set of pixel data, which form the color flow mapped ultrasound image. For example, a color flow image corresponding to the first set of pixel data overlaid on the B-mode image corresponding to the second set of pixel data. In connection with FIG. 4, the controller circuit 136 may select which pixels of the first and second set of pixel data to include in the select pixel data set based on a power threshold and/or a velocity threshold.

FIG. 4 illustrates a flowchart of a method 400 for identifying a select pixel data set, in accordance with various embodiments described herein. The method 400, for example, may employ structures or aspects of various embodiments (e.g., systems and/or methods) discussed herein. In various embodiments, certain steps (or operations) may be omitted or added, certain steps may be combined, certain steps may be performed simultaneously, certain steps may be performed concurrently, certain steps may be split into multiple steps, certain steps may be performed in a different order, or certain steps or series of steps may be re-performed in an iterative fashion. In various embodiments, portions, aspects, and/or variations of the method 400 may be used as one or more algorithms to direct hardware to perform one or more operations described herein. It should be noted, other methods may be used, in accordance with embodiments herein.

Beginning at 402 the controller circuit 136 may receive the velocity threshold and/or the power threshold. The velocity threshold may correspond to a range of velocities to be included and/or not included in a resulting ultrasound image. The power threshold may be based on a signal to noise ratio. Optionally, the power threshold may be based on the power of pulses forming the pulse sequence corresponding to the color flow imaging. For example, the power threshold may be a percentage of the power delivered by the pulse sequence, a set ratio (e.g., dB) between the Doppler power signal and the power of the pulses forming the pulse sequence, and/or the like.

In various embodiments, the controller circuit 136 may receive the velocity threshold and/or the power threshold from the user interface 142. For example, the user may select one or more interface components to define a range of velocities. Additionally or alternatively, the velocity threshold and/or the power threshold may be stored in the memory 140 and accessed and/or received by the controller circuit 136. Optionally, the velocity threshold and/or the power threshold may be received be the controller circuit 136 from a remote system such as a server communicably coupled to the ultrasound imaging system 100.

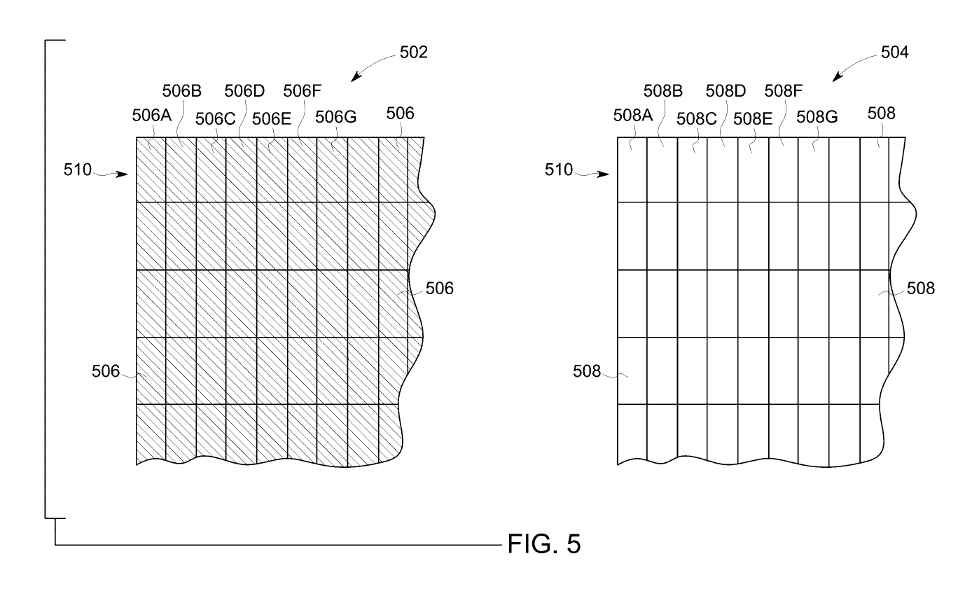

At 404 the controller circuit 136 may select a first pixel location of the first and second set of pixel data. In connection with FIG. 5, a first pixel location 510 may correspond to a pixel from a first set of pixel data 502 and a second set of pixel data 504, which are associated with a single position or location of the ROI.

FIG. 5 illustrates a schematic of a portion of the first set of pixel data 502 and the second set of pixel data 504. The first and second set of pixel data 502 and 504 are illustrated as arrays of pixels 506 (e.g., 506a-e) and 508 (e.g., 508a-e) based on the color flow data 273 and B-mode data 276, respectively. The first pixel location 510 selected by controller circuit 136 may correspond to a location within the ROI, which is represented by one or more of the pixels 506 and 508 in the first set of pixel data 502 and the second set of pixel data 504, respectively. For example, the select pixel location may correspond to the pixel 506a and 508a of the first and second set of pixel data 502 and 504, respectively.

It should be noted that in various embodiments a number of pixels 506 of the first set of pixel data 502 may be the same as and/or different than a number of pixels 508 in the second set of pixel data 504. For example, the B-mode data 276 may include pixels 508 corresponding to anatomical structures surrounding and/or outside of the ROI, which are not a part of the color flow data 273.

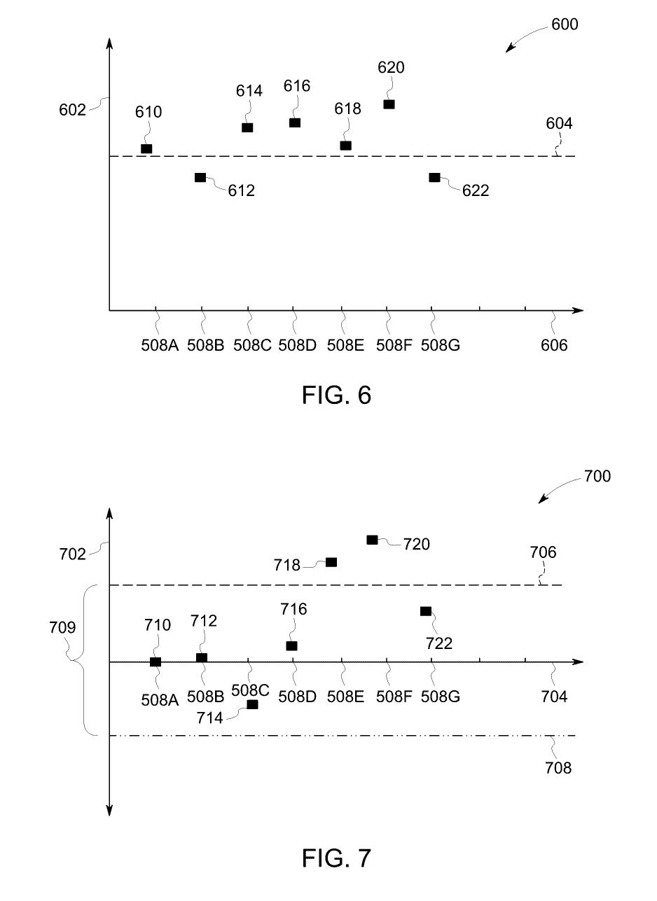

At 406 the controller circuit 136 may compare the Doppler signal power at the select pixel location 510 with the power threshold. In connection with FIG. 6, the controller circuit 136 may compare the Doppler signal power 610 of the pixel 506a, corresponding to the select pixel location 510, with the power threshold 604.

FIG. 6 is a graphical representation 600 of the Doppler signal power 610-622 corresponding to and/or represented by the pixels 506a-506g over a horizontal axis 606 corresponding to a pixel location and a vertical axis 602 representing a magnitude of the Doppler signal. For example, the Doppler signal power 610-622 calculated by the controller circuit 136 at 304 (FIG. 3) may be included within the color flow data 273 stored in the memory 290 with corresponding pixels 506a-506g determined by the scan converter 292.

At 408 the controller circuit 136 determines whether the Doppler signal power is above the power threshold. In connection with FIG. 6, since the Doppler signal power 610 of the pixel 506a is greater than the power threshold 604, the controller circuit 136 may determine that the Doppler signal power 610 is above the power threshold 604.

If the Doppler signal power is above the power threshold, at 410 the controller circuit 136 may compare the flow velocity of the select pixel location with the velocity threshold. In connection with FIG. 7, the controller circuit 136 may compare the flow velocity 710 of the pixel 506a, corresponding to the select pixel location 510, with the velocity threshold 709.

FIG. 7 is a graphical representation 700 of the flow velocity 710-722 of the pixels 506a-506g over a horizontal axis 704 corresponding to a pixel location and a vertical axis 702 representing a flow velocity with respect to the ultrasound probe 126. It should be noted that the horizontal axis 704 is illustrated at an origin or zero flow velocity position of the vertical axis 702. The velocity threshold 709 is illustrated as a range of velocities defined between a negative or first velocity 708 and a positive or second velocity 706. It should be noted in various embodiments the first and second velocities 708 and 706 may not have the same magnitudes. Additionally or alternatively, the velocity threshold 709 may correspond to a flow velocity magnitude not based on a direction of the flow velocity with respect to the ultrasound prove 126 (e.g., negative, positive). In various embodiments, the flow velocities 710-722 calculated by the controller circuit 136 at 304 (FIG. 3) may be included within the color flow data 273 stored in the memory 290 with the corresponding pixels 506a-506g determined by the scan converter 292.

At 412, the controller circuit 136 determines whether the flow velocity is over the velocity threshold. In various embodiments, the controller circuit 136 may determine whether the flow velocity is within the velocity threshold 709, for example, between the first and second velocity 708 and 706 defining the velocity threshold 709. For example, since the flow velocity 710 of the pixel 506a is positioned between the first and second velocities 708 and 706, the controller circuit 136 may determine that the flow velocity 710 is not over the velocity threshold 709.

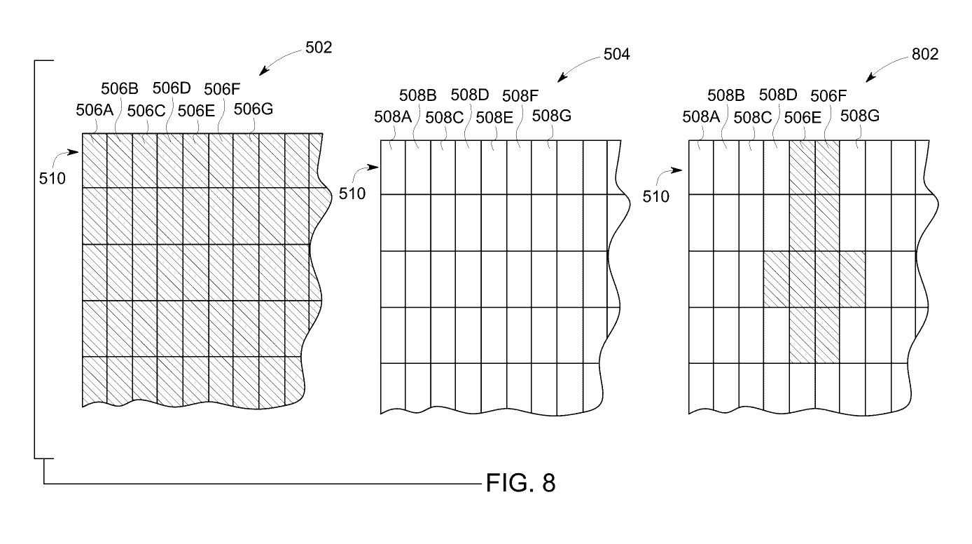

If the Doppler signal power or the flow velocity is below the power threshold or the velocity threshold, respectively, at 414 the controller circuit 136 may add the select pixel location of the second set of pixel data to the select pixel data set. In connection with FIG. 8, since the controller circuit 136 determined that the flow velocity 710 of the pixel 506a is below the velocity threshold 709, the controller circuit 136 may add the pixel 508a of the second set of pixel data 504 to the select pixel data set 802 at the select pixel location 510.

FIG. 8 illustrates a schematic of a portion of the select pixel data set 802 formed by select pixels 506 (e.g., 506e-f) and 508 (e.g., 508a-d, 508g) of the first and second set of pixel data 502 and 504, respectively. The select pixel data set 802 may include pixels of the first set of pixel data 502 and the second set of pixel data 504. For example, the controller circuit 136 may determine that the pixels 506b-d have Doppler signal power 612-616 and/or flow velocities 712-716 below the power threshold 604 and/or velocity threshold 709, respectively, at multiple select pixel locations. Based on the determination by the controller circuit 136, the pixels 508b-d may be added by the controller circuit 136 to the select pixel data set 802.

If the flow velocity is above the velocity threshold, at 416 the controller circuit 136 may add the select pixel location of first set of pixel data to the select pixel data set. For example, the controller circuit 136 may determine that the pixels 506e-f have Doppler signal power 618-620 and flow velocities 718-720 above the power threshold 604 and the velocity threshold 709, respectively at different select pixel locations. Based on the determination by the controller circuit 136, the pixels 506e-f may be added by the controller circuit 136 to the select pixel set 802.

At 418, the controller circuit 136 determines whether additional pixel location are available. In various embodiments, the controller circuit 136 may repeat the threshold operations at 406-416 for multiple pixel locations to form the select pixel data set 802. For example, when the controller circuit 136 determines that a number of pixels of the select pixel data set 802 is less than a number of pixels of the first set of pixel data 502 and/or the second set of pixel data 504, the controller circuit 136 may determine an additional pixel location may be selected and selects one of the remaining or alternative pixel locations at 420 and returns to the operation at 406.

If no additional pixel location are available, at 422 the controller circuit 136 forms the ultrasound image. For example, the select pixel data set 802 may form one or more frames of an ultrasound image of the ROI, such as a color flow mapped ultrasound image, which includes a color flow data and B-mode data. The controller circuit 136 may store the select pixel data set 802 on memory (e.g., the memory 290, the memory 140).

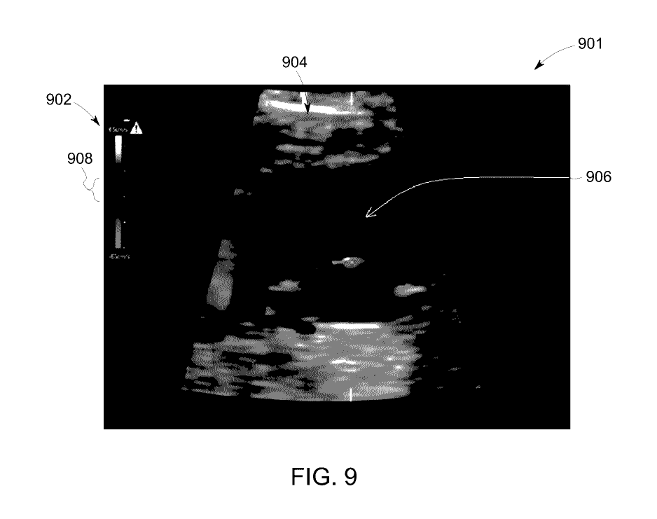

Returning to FIG. 3, at 310, the controller circuit 136 displays the ultrasound image. FIG. 9 illustrates the ultrasound image 901, which may be shown on the display 138. The ultrasound image 901 may correspond to one of the frames formed by the select pixel data set 802 generated by the controller circuit 136 from the color flow data (e.g., corresponding to the pixel group 906) and the B-mode data (e.g., corresponding to the pixel group 904). The flow velocity is represented by a color, such as red and blue, to represent a speed and a direction (e.g., with respect to the ultrasound probe 126) of the flow. The ultrasound image 901 may include a color meter 902 with a defined color spectrum, which relates or associates a pixel color with a corresponding speed (e.g., cm/s) and direction. Optionally, the color meter 902 may include a region 908 representing velocities not included within the color flow data shown on the ultrasound image 901.

In operation, a size and/or position of the region 908 with respect to the color meter 902 may be adjusted by the user. For example, the user may move the region 908 along the color meter 902 and/or adjust a size of the region 908 using the user interface 142. Based on a position and/or size of the region 908 the controller circuit 136 may adjust and/or receive the velocity threshold. In connection with FIG. 7, by increasing a size of the region 908, the controller circuit 136 may increase the range of velocities (e.g., moving the first and/or second velocity 708, 706) defining the velocity threshold 709. When the velocity threshold 709 is adjusted, the controller circuit 136 may repeat one or more operations (e.g., 308-310, 404-422) to generate a new ultrasound image 901, which may include a different number of pixels based on the color flow data (e.g., replacing pixels representing color flow data having flow velocities not over the adjusted velocity threshold with pixels representing B-mode data, replacing pixels representing B-mode data with pixels representing color flow data having flow velocities over the adjusted velocity threshold).

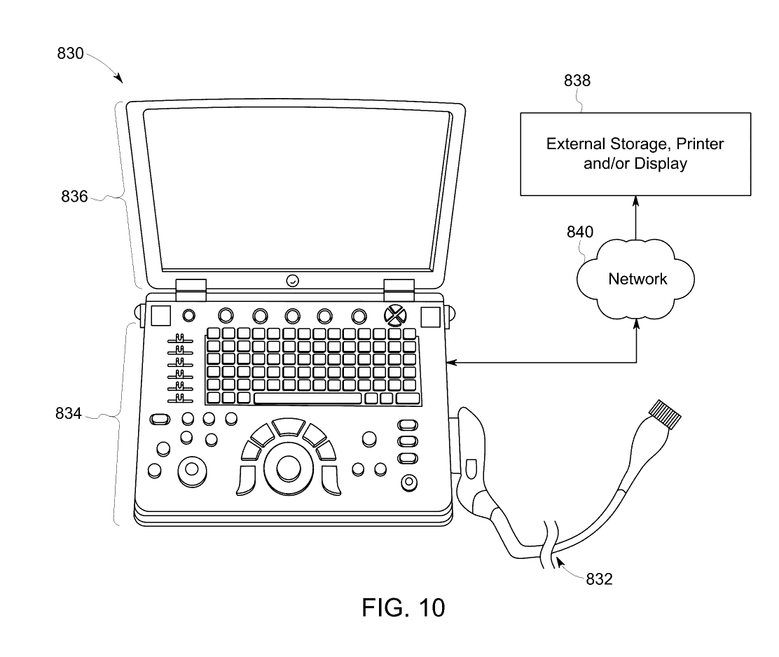

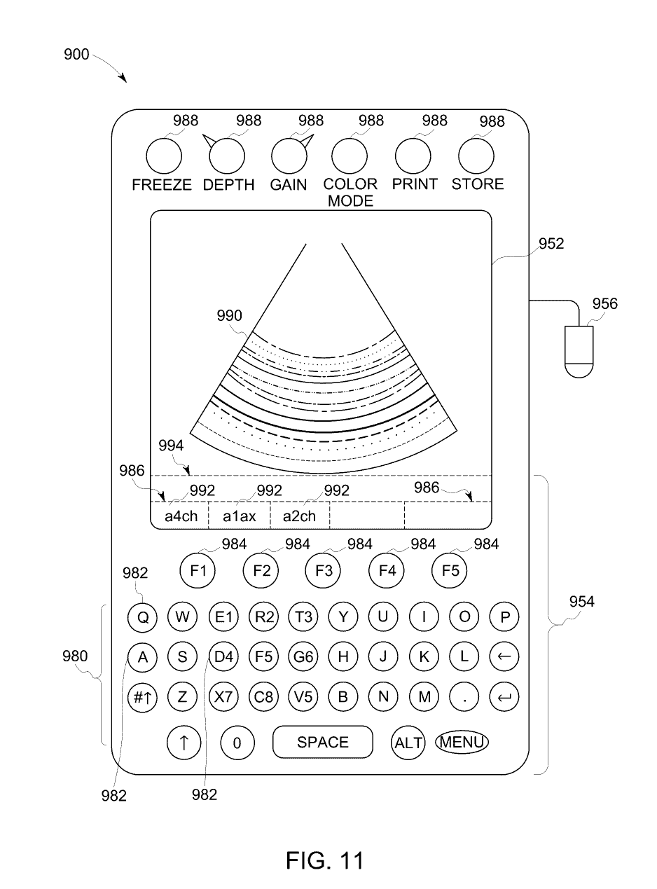

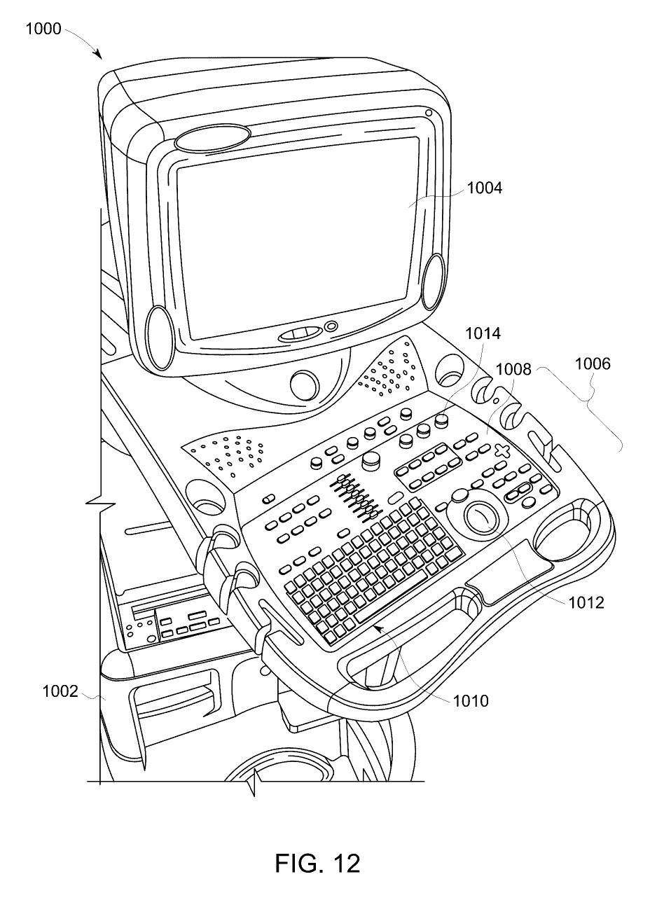

The ultrasound system 100 of FIG. 1 may be embodied in a small-sized system, such as laptop computer or pocket-sized system as well as in a larger console-type system. FIGS. 10 and 11 illustrate small-sized systems, while FIG. 12 illustrates a larger system.

FIG. 10 illustrates a 3D-capable miniaturized ultrasound system 830 having a probe 832 that may be configured to acquire 3D ultrasonic data or multi-plane ultrasonic data. For example, the probe 832 may have a 2D array of elements as discussed previously with respect to the probe. A user interface 834 (that may also include an integrated display 836) is provided to receive commands from an operator. As used herein, “miniaturized” means that the ultrasound system 830 is a handheld or hand-carried device or is configured to be carried in a person's hand, pocket, briefcase-sized case, or backpack. For example, the ultrasound system 830 may be a hand-carried device having a size of a typical laptop computer. The ultrasound system 830 is easily portable by the operator. The integrated display 836 (e.g., an internal display) is configured to display, for example, one or more medical images.

The ultrasonic data may be sent to an external device 838 via a wired or wireless network 840 (or direct connection, for example, via a serial or parallel cable or USB port). In some embodiments, the external device 838 may be a computer or a workstation having a display. Alternatively, the external device 838 may be a separate external display or a printer capable of receiving image data from the hand carried ultrasound system 830 and of displaying or printing images that may have greater resolution than the integrated display 836.

FIG. 11 illustrates a hand carried or pocket-sized ultrasound imaging system 900 wherein the display 952 and user interface 954 form a single unit. By way of example, the pocket-sized ultrasound imaging system 900 may be a pocket-sized or hand-sized ultrasound system approximately 2 inches wide, approximately 4 inches in length, and approximately 0.5 inches in depth and weighs less than 3 ounces. The pocket-sized ultrasound imaging system 900 generally includes the display 952, user interface 954, which may or may not include a keyboard-type interface and an input/output (I/O) port for connection to a scanning device, for example, an ultrasound probe 956. The display 952 may be, for example, a 320×320 pixel color LCD display (on which a medical image 990 may be displayed). A typewriter-like keyboard 980 of buttons 982 may optionally be included in the user interface 954.

Multi-function controls 984 may each be assigned functions in accordance with the mode of system operation (e.g., displaying different views). Therefore, each of the multi-function controls 984 may be configured to provide a plurality of different actions. One or more interface components, such as label display areas 986 associated with the multi-function controls 984 may be included as necessary on the display 952. The system 900 may also have additional keys and/or controls 988 for special purpose functions, which may include, but are not limited to “freeze,” “depth control,” “gain control,” “color-mode,” “print,” and “store.”

One or more of the label display areas 986 may include labels 992 to indicate the view being displayed or allow a user to select a different view of the imaged object to display. The selection of different views also may be provided through the associated multi-function control 984. The display 952 may also have one or more interface components corresponding to a textual display area 994 for displaying information relating to the displayed image view (e.g., a label associated with the displayed image).

It should be noted that the various embodiments may be implemented in connection with miniaturized or small-sized ultrasound systems having different dimensions, weights, and power consumption. For example, the pocket-sized ultrasound imaging system 900 and the miniaturized ultrasound system 830 may provide the same scanning and processing functionality as the system 100.

FIG. 12 illustrates an ultrasound imaging system 1000 provided on a movable base 1002. The portable ultrasound imaging system 1000 may also be referred to as a cart-based system. A display 1004 and user interface 1006 are provided and it should be understood that the display 1004 may be separate or separable from the user interface 1006. The user interface 1006 may optionally be a touchscreen, allowing the operator to select options by touching displayed graphics, icons, and the like.

The user interface 1006 also includes control buttons 1008 that may be used to control the portable ultrasound imaging system 1000 as desired or needed, and/or as typically provided. The user interface 1006 provides multiple interface options that the user may physically manipulate to interact with ultrasound data and other data that may be displayed, as well as to input information and set and change scanning parameters and viewing angles, etc. For example, a keyboard 1010, trackball 1012 and/or multi-function controls 1014 may be provided.

It should be noted that the various embodiments may be implemented in hardware, software or a combination thereof. The various embodiments and/or components, for example, the modules, or components and controllers therein, also may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor further may include a storage device, which may be a hard disk drive or a removable storage drive such as a solid-state drive, optical disk drive, and the like. The storage device may also be other similar means for loading computer programs or other instructions into the computer or processor.

As used herein, the term “computer,” “subsystem” or “module” may include any processor-based or microprocessor-based system including systems using microcontrollers, reduced instruction set computers (RISC), ASICs, logic circuits, and any other circuit or processor capable of executing the functions described herein. The above examples are exemplary only, and are thus not intended to limit in any way the definition and/or meaning of the term “computer”.

The computer or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer or processor as a processing machine to perform specific operations such as the methods and processes of the various embodiments. The set of instructions may be in the form of a software program. The software may be in various forms such as system software or application software and which may be embodied as a tangible and non-transitory computer readable medium. Further, the software may be in the form of a collection of separate programs or modules, a program module within a larger program or a portion of a program module. The software also may include modular programming in the form of object-oriented programming. The processing of input data by the processing machine may be in response to operator commands, or in response to results of previous processing, or in response to a request made by another processing machine.

As used herein, a structure, limitation, or element that is “configured to” perform a task or operation is particularly structurally formed, constructed, or adapted in a manner corresponding to the task or operation. For purposes of clarity and the avoidance of doubt, an object that is merely capable of being modified to perform the task or operation is not “configured to” perform the task or operation as used herein. Instead, the use of “configured to” as used herein denotes structural adaptations or characteristics, and denotes structural requirements of any structure, limitation, or element that is described as being “configured to” perform the task or operation. For example, a controller circuit, processor, or computer that is “configured to” perform a task or operation may be understood as being particularly structured to perform the task or operation (e.g., having one or more programs or instructions stored thereon or used in conjunction therewith tailored or intended to perform the task or operation, and/or having an arrangement of processing circuitry tailored or intended to perform the task or operation). For the purposes of clarity and the avoidance of doubt, a general purpose computer (which may become “configured to” perform the task or operation if appropriately programmed) is not “configured to” perform a task or operation unless or until specifically programmed or structurally modified to perform the task or operation.

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the various embodiments without departing from their scope. While the dimensions and types of materials described herein are intended to define the parameters of the various embodiments, they are by no means limiting and are merely exemplary. Many other embodiments will be apparent to those of skill in the art upon reviewing the above description. The scope of the various embodiments should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. §112(f) unless and until such claim limitations expressly use the phrase “means for” followed by a statement of function void of further structure.

This written description uses examples to disclose the various embodiments, including the best mode, and also to enable any person skilled in the art to practice the various embodiments, including making and using any devices or systems and performing any incorporated methods. The patentable scope of the various embodiments is defined by the claims, and may include other examples that occur to those skilled in the art. Such other examples are intended to be within the scope of the claims if the examples have structural elements that do not differ from the literal language of the claims, or the examples include equivalent structural elements with insubstantial differences from the literal language of the claims.

What is claimed is:

1. A method for generating an ultrasound image, the method comprising:
collecting ultrasound data for a volumetric region of interest (ROI), wherein the ultrasound data includes color flow data and B-mode image data;
calculating a Doppler signal power and a flow velocity based on the color flow data corresponding to pixels within a first set of pixel data;
generating a second set of pixel data based on the B-mode image data;
identifying a select pixel data set from the first set of pixel data and the second set of pixel data based on the Doppler signal power and the flow velocity, wherein the select pixel data forms an ultrasound image of the ROI; and
displaying the ultrasound image on a display.

2. The method of claim 1, further comprising:
comparing the Doppler signal power to a power threshold; and
comparing the flow velocity to a velocity threshold, wherein the select pixel data set includes pixels of the first set of pixel data and the second set of pixel data based on the power threshold and the velocity threshold.

3. The method of claim 2, further comprising receiving the velocity threshold from a user interface.

4. The method of claim 2, wherein the velocity threshold corresponds to a range of velocities.

5. The method of claim 2, wherein the power threshold is based on a signal to noise ratio.

6. The method of claim 1, further comprising receiving a velocity threshold from a user interface, wherein the select pixel data includes pixels of the first set of pixel data and the second set of pixel data further based on the velocity threshold.

7. The method of claim 1, wherein the select pixel data includes pixels from the first set of pixel data and the second set of pixel data.

8. An ultrasound imaging system for a mean velocity comprising:
an ultrasound probe configured to acquire ultrasound data of a patient;
a display;
a memory configured to store programmed instructions; and
one or more processors configured to execute the programmed instructions stored in the memory, wherein the one or more processors when executing the programmed instructions perform the following operations:
collect the ultrasound data from the ultrasound probe for a volumetric region of interest (ROI), wherein the ultrasound data includes color flow data and B-mode image data;
calculate a Doppler signal power and a flow velocity based on the color flow data corresponding to pixels within a first set of pixel data;
generate a second set of pixel data based on the B-mode image data;
identify a select pixel data set from the first set of pixel data and the second set of pixel data based on the Doppler signal power and the flow velocity, wherein the select pixel data forms an ultrasound image of the ROI; and
display the ultrasound image on the display.

9. The ultrasound imaging system of claim 8, wherein the one or more processors further:
compare the Doppler signal power to a power threshold; and
compare the flow velocity to a velocity threshold, wherein the select pixel data set includes pixels of the first set of pixel data and the second set of pixel data based on the power threshold and the velocity threshold.

10. The ultrasound imaging system of claim 9, wherein the one or more processors further receive the velocity threshold from a user interface.

11. The ultrasound imaging system of claim 9, wherein the velocity threshold corresponds to a range of velocities.

12. The ultrasound imaging system of claim 9, wherein the power threshold is based on a signal to noise ratio.

13. The ultrasound imaging system of claim 8, further comprising a user interface, wherein the one or more processors further receive a velocity threshold from the user interface, the select pixel data including pixels of the first set of pixel data and the second set of pixel data based on the velocity threshold.

14. The ultrasound imaging system of claim 8, wherein the select pixel data includes pixels from the first set of pixel data and the second set of pixel data.

15. A tangible and non-transitory computer readable medium comprising one or more computer software modules configured to direct one or more processors to:
collect ultrasound data for a volumetric region of interest (ROI), wherein the ultrasound data includes color flow data and B-mode image data;
calculate a Doppler signal power and a flow velocity based on the color flow data corresponding to pixels within a first set of pixel data;
generate a second set of pixel data based on the B-mode image data;
identify a select pixel data set from the first set of pixel data and the second set of pixel data based on the Doppler signal power and the flow velocity, wherein the select pixel data forms an ultrasound image of the ROI; and
display the ultrasound image on a display.

16. The tangible and non-transitory computer readable medium of claim 15, wherein the one or more processors are further directed to compare the Doppler signal power to a power threshold, and compare the flow velocity to a velocity threshold, the select pixel data set including pixels of the first set of pixel data and the second set of pixel data based on the power threshold and the velocity threshold.

17. The tangible and non-transitory computer readable medium of claim 16, wherein the one or more processors are further directed to receive the velocity threshold from a user interface.

18. The tangible and non-transitory computer readable medium of claim 16, wherein the velocity threshold corresponds to a range of velocities.

19. The tangible and non-transitory computer readable medium of claim 16, wherein the power threshold is based on a signal to noise ratio.

20. The tangible and non-transitory computer readable medium of claim 15, wherein the select pixel data includes pixels from the first set of pixel data and the second set of pixel data.
